(19) United States
(12) Patent Application Publication
(10) Pub. No.: US 2022/0192533 A1
(43) Pub. Date: Jun. 23, 2022

(54) SWALLOWABLE DEVICE FOR SLEEP APNEA DETECTION

(71) Applicant: Celero Systems, Inc., Lincoln, MA (US)

(72) Inventors: Benjamin David Pless, Lincoln, MA (US); Daniel Bacher, Walpole, MA (US)

(21) Appl. No.: 17/553,970

(22) Filed: Dec. 17, 2021

Related U.S. Application Data
(60) Provisional application No. 63/126,634, filed on Dec. 17, 2020.

(51) Int. Cl. A61B 5/07; A61B 5/00

(52) U.S. Cl. CPC A61B 5/073; A61B 2562/0271; A61B 2562/0219; A61B 5/4818

(57) ABSTRACT

A swallowable device to detect sleep apnea in a patient in need thereof. The swallowable device includes an ingestible capsule. An accelerometer is contained within the ingestible capsule and is configured to reside within the stomach to detect a respiration parameter(s) indicative of sleep apnea and generate a sensing signal based thereon. The ingestible capsule also includes a radio contained within the ingestible capsule and a micro-controller unit (MCU) in electrical communication with the accelerometer and the radio and configured and programmed to receive the sensing signal. The ingestible capsule can also include a microphone to detect a respiration parameter(s) indicative of sleep apnea and/or snoring.

### CROSS-REFERENCE TO RELATED APPLICATIONS

The present application claims priority to U.S. Provisional Application No. 63/126,634, filed on Dec. 17, 2020, which is incorporated herein by reference in its entirety.

### TECHNICAL FIELD

The present application relates to an ingestible capsule for sleep apnea detection.

### BACKGROUND

Sleep apnea is a condition marked by abnormal breathing during sleep. People with sleep apnea have multiple extended pauses in breath when they sleep. These temporary breathing lapses cause lower-quality sleep and affect the body's supply of oxygen, leading to potentially serious health consequences such as high blood pressure, heart attack, heart disease, and stroke.

Unfortunately, sleep disorders are notoriously underdiagnosed because they often develop gradually over time and have symptoms that are difficult to recognize. Accurate diagnosis is necessary to enable effective treatment for sleep disorders. A sleep study provides valuable insight into the causes of a person's symptoms including which, if any, sleep disorder is present. Sleep studies are also used in people diagnosed with sleep disorders to monitor their response to treatment.

Polysomnography (i.e., a sleep study) records certain body functions as a person sleeps. These studies are usually done at a sleep lab which can be inconvenient for some people. At home sleep apnea tests/studies are available to evaluate for obstructive sleep apnea (OSA) as they are less inconvenient than polysomnography in a sleep lab.

A home sleep apnea test is a very simplified breathing monitor that typically tracks breathing-oxygen levels, and breathing effort while being worn during sleep. It often requires a finger probe to measures oxygen levels, a mask with tubes that extend to the patient's nostrils and sensors applied to their abdomen and chest to measure its rise and fall as the patient breathes. The equipment used can be expensive and interfere with normal sleeping. A convenient, more reliable, less cumbersome approach is needed for at home sleep tests.

### SUMMARY

A swallowable device to detect sleep apnea in a patient in need thereof is provided. The device can comprise an ingestible capsule comprising a housing having at least one retention arm extending from at least one side surface thereof. The at least one retention arm is sized and configured to retain the housing within the stomach such that the housing does not pass to the duodenum. An accelerometer can be contained within the ingestible capsule to detect at least a first parameter indicative of sleep apnea and generate at least a first sensing signal based thereon. The ingestible capsule can also include a radio for communication purposes. A micro-controller unit (MCU) can be contained within the ingestible capsule and can be in electrical communication with the accelerometer and the radio and can be configured and programmed to receive the at least first sensing signal. The MCU can be configured and programmed to deliver at least a first communication signal via the radio to an external base station in response to the at least first sensing signal. The at least first parameter can comprise any suitable parameter indicative of sleep apnea such as, for example, low minute ventilation, respiration rate, respiratory effort, a sleep apnea event, or a combination thereof. The radio can be configured to communicate with the external base station and the MCU can be configured and programmed to control the operation of the radio to send the at least first communication signal to the external base station in response to the at least first sensing signal. The swallowable device can further include an energy source configured to provide electrical power to operate the controller, the accelerometer, the radio, or a combination thereof.

The ingestible device can also include a microphone/acoustic sensor that can be configured to detect an at least second parameter indicative of sleep apnea and generate an at least second sensing signal based thereon. The MCU in electrical communication with the microphone and configured and programmed to receive the at least second sensing signal. The MCU can be configured and programmed to deliver an at least second communication signal via the radio to an external base station in response to the at least second sensing signal. The at least second parameter can be any suitable parameter indicative of sleep apnea such as, for example, breathing effort, snoring, or both. The MCU can be configured and programmed to analyze the at least second respiration parameter to extract data on snoring and respiratory effort of the patient.

For example, the swallowable device can save sensed data from the sensors (e.g. accelerometer, acoustic sensor, etc) and the data can be stored in memory and then, independent of the timing the sensed data is obtained, the data can be uploaded to an external base station. The data can be uploaded at the same or different time as the sensed information is obtained. For example, a sleep study patient could swallow the device at night and then the data could be uploaded the next day.

The MCU can be configured and programmed to distinguish individual breaths of the patient and to control operation of the radio to send a time series or waveform of breath-to-breath intervals, or a combination thereof to an external base station. The MCU can also be configured and programmed to generate a set of at least one feature for a predictive model from at least the first parameter. For example, the MCU can be configured and programmed to generate the set of at least one feature for a predictive model from the at least a first parameter detected by the accelerometer and the at least second parameter detected at the microphone. The MCU can be configured and programmed to implement the predictive model. The predictive model can provide a clinical parameter representing one of apnea, hypopnea, and snoring associated with the patient from the set of at least one feature. In particular, the MCU can be configured and programmed to transmit the features and the timing of the features derived from the feature extraction to an external base station. The external base station can be configured and programmed to implement the predictive model. The predictive model can provide a clinical parameter representing one of apnea, hypopnea, and snoring associated with the patient from the set of at least one feature.

The ingestible capsule can also include other sensors, such as a temperature sensor.

### BRIEF DESCRIPTION OF DRAWINGS

FIG. 1 is a diagram of a swallowable device for detecting sleep apnea according to an aspect of the present disclosure.

FIG. 2 is a side view of a swallowable device for detecting sleep apnea according to an aspect of the present disclosure.

FIG. 3 is a perspective view of the swallowable device of FIG. 2.

### DETAILED DESCRIPTION

As used herein with respect to a described element, the terms “a,” “an,” and “the” include at least one or more of the described elements including combinations thereof unless otherwise indicated. Further, the terms “or” and “and” refer to “and/or” and combinations thereof unless otherwise indicated. By “substantially” is meant that the shape or configuration of the described element need not have the mathematically exact described shape or configuration of the described element but can have a shape or configuration that is recognizable by one skilled in the art as generally or approximately having the described shape or configuration of the described element. A “patient” as described herein includes a mammal, such as a human being. An “ingestible capsule” as used herein is a capsule that is not just capable of being ingested but rather is suitable for swallowing and entering into the gastrointestinal tract. The terms “first” and “second” are not used in a quantitative sense but rather to distinguish one element from another element unless otherwise indicated.

A swallowable device is provided for simple, convenient, home monitoring of sleep patterns overnight, as well as follow-up monitoring after initiation of therapeutic interventions such as continuous positive airway pressure (CPAP). An advantage of devices as disclosed herein is that it is easy to implement (e.g. a patient can take a pill at bedtime), it allows multi-day or repeat studies over a period of time to more fully assess the degree of apnea, and to monitor the progress of therapies to treat sleep apnea.

In an aspect, a swallowable device is provided with a pill-like form factor, such as an ingestible capsule, suitable for swallowing. Contained within the ingestible capsule, is a miniaturized circuit that contains an accelerometer, a micro-controller unit (MCU), a radio, and an optional microphone. Once ingested, the miniaturized circuit can monitor respiratory parameters such as low minute ventilation that would include many breaths, respiratory rate, respiratory effect and/or sleep apnea events and can use auditory information to monitor breathing effort and/or snoring as the device travels through the digestive system and/or while residing in the stomach.

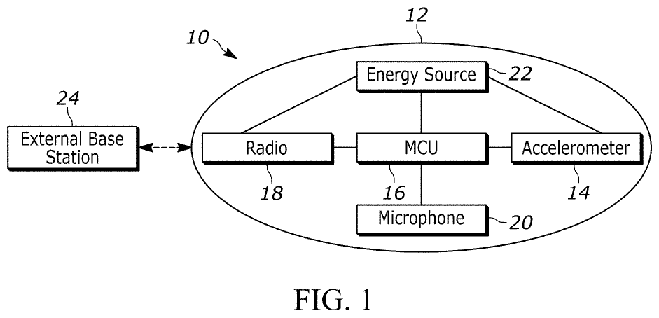

As shown in FIG. 1 the device 10 can comprise an ingestible capsule 12, an accelerometer 14 in electrical connection with a micro controller unit (MCU) 16, which is in electrical connection with a radio 18 and an optional microphone 20. The electronic circuitry can be in electrical connection with an energy source 22. The capsule 12 can have a size, shape and weight that can be a suitable predefined size, shape and weight as desired as long as it can contain the required electronics and is made of suitable material(s) for an ingestible medical diagnostic device. Various sizes, shapes and weights may be desired as a controlled input variable for motional and dynamics orientation characterization of the device in the digestive track, such as the stomach, but preferably the device is the size of a standard 00 capsule or smaller. The weight, size and shape of the device can be designed to various specifications for evaluation of motility, motion and orientation characterization with different physical attributes. For example, the device may be round, oval, rectangular or any other shape with varied weight and balance.

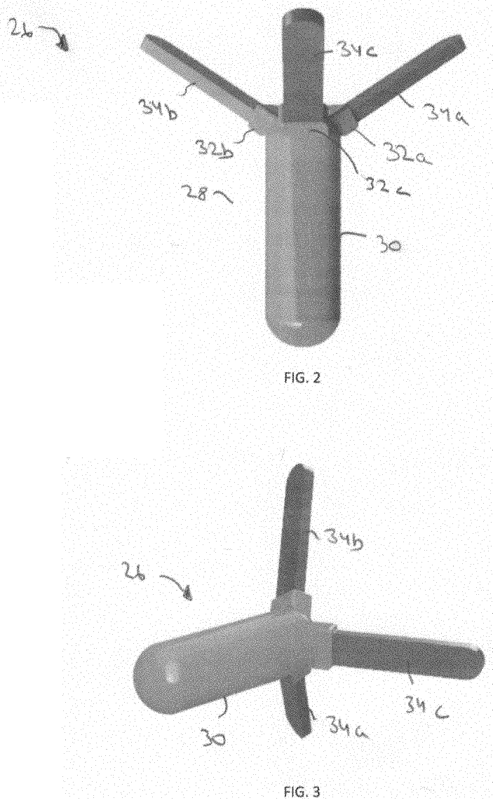

Referring to FIGS. 2 and 3, an ingestible capsule 28 of a swallowable device 26 can comprise a housing 30 having at least one retention arm 34 extending from at least one side surface 32 thereof. The at least one retention arm 34 is sized and configured to retain housing 30 within the stomach such that the housing does not pass to the duodenum.

The MCU comprises suitable electronic functions for acceleration sensor sampling and data capture; memory storage; and control and synchronization of other peripheral circuitry. The MCU can receive physiological data from the accelerometer and the microphone. The MCU can perform feature extraction of the accelerometer data to distinguish individual breaths and can use wireless communication to send a time series or waveform of breath-to-breath intervals to an external base-station/external device. The MCU can also analyze audio information from the microphone to extract information on snoring and respiration effort which can then be transmitted using wireless communication to and external base-station/external device 26. Snoring may be monitored in a serial fashion to determine if a therapeutic intervention is reducing the degree of snoring. As the device passes through the digestive system or resides in the stomach, the measurement data can be transferred to the external base station/external device which may be a computer-based device, such as a laptop computer, a desktop computer, a netbook, a computer tablet such as an Interactive Personal Application Device (iPad), a smartphone or any other processor-based device. The external base station may also be a custom electronic device that acts as an intermediary to transmit data from the device to the aforementioned computer device.

In an aspect, the ingested MCU provides feature extraction of respiration rate and respiratory effort and/or snoring, with the features and their timing being transmitted to an external base station for analysis. In other aspects, the ingested MCU performs just one of the aforementioned analyses or combinations thereof. The analysis can use machine learning to assess the degree of apnea, hypopnea and snoring from the data streams whether it is done in the ingested device or in the external base station. The data preferably is presented to the clinician as an Apnea-Hyponia Index (AHI) where, for example, an AHI of less than 5 is normal, 5 to 15 is mild sleep apnea, 15 to 30 is moderate sleep apnea, and greater than 30 is severe sleep apnea.

The radio can use RF communication, infrared light, acoustic (including ultrasonic), conducted electronic, magnetic or any other of wireless signal that can propagate a signal or field through human or animal tissue. If using wireless RF communication, the radio can be implemented with suitable RF integrated circuits that can implement a variety of standard protocols including but not limited to sub-GHz protocols such as 400-433 MHz MICS or 868-915 MHz ISM, and 2.4 GHz Bluetooth or Bluetooth Low Energy.

The energy source for the device can be from a conventional battery (for example a watch battery), a biogalvanic battery, external power transmitted to the device in the GI tract, such as the stomach by RF; infrared light; ultrasound; energy harvesting, other suitable sources; or a combination of sources or methodologies.

Although the present invention provides that the ingestible capsule can contain an accelerometer and a microphone, the capsule can additionally or alternatively include other types of sensors, such as temperature sensors to measure other types of measurable parameters Exemplary parameters and exemplary sensors are provided in Table I.

TABLE I

| Parameters Indicative of Sleep Apnea | Sensors required to measure |
|---|---|
| Respiratory rate | Accelerometer/Acoustic Sensor |
| Respiratory effort | Accelerometer/Acoustic Sensor |
| Minute ventilation | Accelerometer/Acoustic Sensor |
| Apnea and hypopnea events | Accelerometer/Acoustic Sensor |
| Apnea hypopnea index (AHI) | Accelerometer/Acoustic Sensor |
| Core body temperature | Temperature Sensor |

Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

What is claimed is:

1. A swallowable device to detect sleep apnea in a patient in need thereof comprising:
an ingestible capsule comprising a housing having at least one retention arm extending from at least one side surface thereof, the at least one retention arm sized and configured to retain the housing within the stomach;
an accelerometer contained within the ingestible capsule to detect at least a first parameter indicative of sleep apnea and generate at least a first sensing signal based thereon;
a radio contained within the ingestible capsule; and
a micro-controller unit (MCU) in electrical communication with the accelerometer and the radio and configured and programmed to receive the at least first sensing signal.

2. The swallowable device of claim 1, wherein the MCU is configured and programmed to deliver at least a first communication signal via the radio to an external base station in response to the at least first sensing signal.

3. The swallowable device of claim 1, wherein the at least first parameter comprises low minute ventilation, respiration rate, respiratory effort, a sleep apnea event, or a combination thereof.

4. The swallowable device of claim 2, wherein the radio is configured to communicate with the external base station and wherein the MCU is configured and programmed to control the operation of the radio to send the at least first communication signal to the external base station in response to the at least first sensing signal.

5. The swallowable device of claim 2, wherein the MCU is configured and programmed to distinguish individual breaths of the patient and to control operation of the radio to send a time series or waveform of breath-to-breath intervals, or a combination thereof to the external base station.

6. The swallowable device of claim 1, further comprising a microphone contained within the ingestible capsule.

7. The swallowable device of claim 6, wherein the microphone is configured to detect an at least second parameter indicative of sleep apnea and generate an at least second sensing signal based thereon, the MCU in electrical communication with the microphone and configured and programmed to receive the at least second sensing signal.

8. The swallowable device of claim 6, wherein the MCU is configured and programmed to deliver an at least second communication signal via the radio to an external base station in response to the at least second sensing signal.

9. The swallowable device of claim 7, wherein the at least second parameter is breathing effort, snoring, or both.

10. The swallowable device of claim 7, wherein the MCU is configured and programmed to analyze the at least second parameter to extract data on snoring and respiratory effort of the patient.

11. The swallowable device of claim 1, wherein the MCU is configured and programmed to generate a set of at least one feature for a predictive model from at least the first parameter.

12. The swallowable device of claim 7, wherein the MCU is configured and programmed to generate the set of at least one feature for a predictive model from the at least a first parameter detected by the accelerometer and the at least second parameter detected at the microphone.

13. The swallowable device of claim 12, wherein the MCU is configured and programmed to implement the predictive model, the predictive model providing a clinical parameter representing one of apnea, hypopnea, and snoring associated with the patient from the set of at least one feature.

14. The swallowable device of claim 12, wherein the MCU is configured and programmed to transmit the features and the timing of the features derived from the feature extraction to an external base station, the external base station being configured and programmed to implement the predictive model, the predictive model providing a clinical parameter representing one of apnea, hypopnea, and snoring associated with the patient from the set of at least one feature.

15. The swallowable device of claim 1, further comprising an energy source configured to provide electrical power to operate the controller, the accelerometer, the radio, or a combination thereof.

16. The swallowable device of claim 1, wherein the MCU comprises electronics configured for acceleration sensor sampling and data capture; memory storage; and control and synchronization of other peripheral circuitry.

17. The swallowable device of claim 1, further comprising a temperature sensor contained within the ingestible capsule.
